(19) United States
(12) Patent Application Publication
(10) Pub. No.: US 2004/0064152 A1
(43) Pub. Date: Apr. 1, 2004

(54) Device, system, and method for cryosurgical treatment of cardiac arrhythmia

(76) Inventor: Roni Zvuloni, Haifa (IL)

Correspondence Address: Anthony Castorina, G E Ehrlich (1995) Ltd, Suite 207, 2001 Jefferson Davis Highway, Arlington, VA, 22202, US

(21) Appl. No.: 10/311,315

(22) PCT Filed: Sep. 29, 2002
(86) PCT No.: PCT/IL02/00794

(51) Int. Cl. A61N 1/00

(52) U.S. Cl. 607/1

(57) ABSTRACT

The present invention is of systems, devices, and methods for cryogenic treatment of cardiac arrhythmia. More particularly, the present invention is of cryoprobes cooled by Joule-Thomson cooling and having particularized shapes of treatment heads, adapted and adaptable to specific loci of treatment of cardiac arrhythmia. The present invention is further of cryogenic methods for treating cardiac arrhythmia comprising three successive stages of cooling.

### FIELD AND BACKGROUND OF THE INVENTION

The present invention relates to systems, devices, and methods for cryogenic treatment of cardiac arrhythmia. More particularly, the present invention relates to cryoprobes cooled by Joule-Thomson cooling and having particularized shapes of treatment heads, adapted and adaptable to specific loci of treatment of cardiac arrhythmia. The present invention further relates to cryogenic methods for treating cardiac arrhythmia comprising three successive stages of cooling.

Atrial fibrillation is the most common cardiac arrhythmia. Prevalence of atrial fibrillation increases with age, with two cases per thousand at the age of 20-35, increasing to thirty per thousand between the ages of 55 and 60, and to from eighty to a hundred per thousand by age 80.

Thus, at least 4% of the population suffers from atrial fibrillation, and more than 70% of the sufferers are over 65 years old.

Patients with atrial fibrillation have a five-fold increased risk of stroke when compared with normal individuals.

Research has shown that pharmacological approaches to atrial fibrillation have, at one year of treatment, only about 50% success.

In atrial fibrillation and in cardiac arrhythmias in general, pathological electrically transmissive pathways exist within myocardial tissues.

Surgical treatment of arrhythmias seeks to destroy those pathways, thereby preventing transmission of aberrant electrical impulses, and thereby preventing non-synchronized atrial and ventricular contractions.

Popular techniques for treating arrhythmia include methods of cutting or burning lesions in myocardial tissue, preventing electrical conduction therein.

U.S. Pat. No. 6,161,543 to Cox et. al. presents several well-known and widely used techniques, in particular the “MAZE” method.

Currently the MAZE III operation is the most effective treatment of atrial fibrillation, known to have the best long-term success rate. The MAZE procedure pioneered by J. Cox and colleagues creates lines of conduction block that interrupt all potential macro reentrant circuits and cure the atrial fibrillation. The MAZE 3 procedure involves the excision of the atrial appendages, isolation of the pulmonary veins and fragmentation of the atrium, to destroy, and prevent the re-formation of, re-entrant circuits.

The Maze procedure, however, is difficult to execute, and requires a major intervention with consequent complexities of management and often difficult recoveries.

Indeed, all treatment procedures requiring open chest surgery, and particular procedures requiring open-heart surgery and/or heart-lung machine support, are relatively difficult, dangerous, and expensive operations, requiring highly trained practitioners and specialized equipment. They are, moreover, procedures which themselves create major trauma to the patient, cause significant suffering, and are generally followed by long and difficult convalescence.

Consequently, there is a widely recognized need for, and it would be highly advantageous to have, a minimally invasive technique for creation of lesions capable of blocking pathological electrical conduction in atrial tissue, thereby permitting treatment of atrial fibrillation and of other forms of cardiac arrhythmias, yet which does not require subjecting a patient to the trauma of open chest and open heart surgeries.

Techniques for creating the required lesions while avoiding open-heart surgery have been evolved. These include small intercostal percutaneous penetration into the body cavity, endovascular trans-catheter approaches, and others. One popular technique is the use of what is known as a “purse string” procedure to enable a surgeon to practice an opening in an atrial wall, insert a surgical tool, and cut, burn, or freeze tissues therein, while yet allowing continued functioning of the heart.

“Beating heart” surgeries, however, carry with them an intrinsic difficulty. Even for the best of surgeons, it is extremely difficult to position a therapeutic probe in the correct spot for a treatment, and to keep the probe in place during the duration required for a treatment procedure, when that spot is a constantly moving target, a selected tissue on or within a beating heart.

Consequently, there is a widely recognized need for, and it would be highly advantageous to have, a therapeutic device and method enabling to place a therapeutic probe in or on a selected portion of a beating heart, and to maintain that probe accurately in place for a required duration of treatment, without resorting to heart immobilization.

In recent practice, loci in the pulmonary veins are accepted by expert cardiologists as a target for treatment of cardiac arrhythmias. Left atrial muscle fibers are known to penetrate the pulmonary veins, especially the superior pulmonary vein. Pace-maker type cells have been found within these structures, supporting the hypothesis that such structures are a source of ectopic activity and a substrate for multiple re-entry circuits leading to the formation of atrial tachycardia. It is known that persistent atrial tachycardia will cause atrial electrical remodeling, and initiate atrial fibrillation.

Consequently, the pulmonary vein entrance to the atrium has become a locus of a variety of treatment methodologies. However, current techniques using radio frequency energy and high-intensity focused ultrasound to ablate the pulmonary veins orifices are difficult to use successfully, due to inaccurate ablation of tissues in a constantly beating heart, and to inadequate achievement of transmurality.

Thus, there is a widely felt need for, and it would be highly advantageous to have, techniques for creating a circumferential conduction block in a pulmonary vein ostium, which techniques are minimally invasive, minimally traumatic, and which produce lesions sufficiently wide and deep to create a conductive block, yet which do not substantially disturb nor destroy the structural integrity of the atria.

Cryogenic techniques have been used in the field of arrhythmia treatment primarily to effect atrial mapping. Atrial mapping is a procedure utilizing cooling and freezing of tissues to create a temporary blockage of electrical conduction therein. According to atrial mapping procedure, a tissue is selected for inspection and is cooled to a temperature sufficient to temporarily block electrical conductivity, and then the effect of this blockage on the patient's heart rhythms is observed. In this manner, it is possible to map regions responsible for aberrant electrical pulses and non-synchronized contractions, since when such a region is thus cooled, arrhythmia is reduced or abolished.

Atrial mapping, however, is a long and slow procedure. Moreover, currently accepted therapeutic techniques utilize cryogenic mapping to map areas responsible for pathological conduction, and then utilize a separate technique, such as ablation by laser, by radio frequency energy, or by high-intensity focused ultrasound, to ablate the pathological tissues.

Thus, there is a widely felt need for, and it would be highly advantageous to have, a device and method for combining mapping of pathological areas and treatment of those pathological areas in a single coordinated technique. It would be yet further advantageous if such a coordinated technique guaranteed a high degree of reliability in ensuring that the problematic locations identified by mapping are indeed the locations subsequently subject to ablation.

### SUMMARY OF THE INVENTION

According to one aspect of the present invention there is provided a form-fitting cryoprobe having a treatment head sized and formed to fit a shape of a specific organic cryoablation target, said treatment head comprising a Joule-Thomson cooler operable to cool said treatment head, and optionally comprising a Joule-Thomson heater to heat said treatment head.

According to another aspect of the present invention there is provided a shape-adaptable cryoprobe having a treatment head operable to conform to a shape of a cryoablation target, said treatment head comprising a Joule-Thomson cooler operable to cool said treatment head, and preferably a Joule-Thomson heater to heat said treatment head.

According to yet another aspect of the present invention there is provided a cryoprobe for cryogenic treatment of cardiac arrhythmia, said cryoprobe comprising:

a) a form-fitting treatment head sized and shaped to fit a pulmonary vein ostium;

b) a Joule-Thomson cooler operable to cool said treatment head.

According to further features in preferred embodiments of the invention described below, the cryoprobe further comprises a Joule-Thomson heater operable to heat said treatment head, a gas input lumen operable to supply compressed cooling gas to the treatment head; and a gas exhaust lumen operable to exhaust gas from the treatment head.

According to still further features in the described preferred embodiments, the cryoprobe further comprises a plurality of gas input lumens and supply of gas to each of the plurality of gas input lumens is operable to be individually controlled.

According to still further features in the described preferred embodiments, the treatment head further comprises a Joule-Thomson orifice, a heat exchanging configuration, and an active cooling module on a distal face of the treatment head. The active cooling module is operable to create a temporary conduction block in a pulmonary vein ostium, and to create a permanent conduction block in a pulmonary vein ostium.

According to still further features in the described preferred embodiments, the active cooling module is further operable to heat tissues of a pulmonary vein ostium.

According to still further features in the described preferred embodiments, the cryoprobe further comprises a plurality of active cooling modules on the distal face of the treatment head, which may be radially distributed or circumferentially distributed. Each of the plurality of active cooling modules is in fluid communication with an independently controlled source of cooling gas.

According to still further features in the described preferred embodiments, supply of gas to each of a plurality of gas input lumens is operable to be individually controlled.

According to still further features in the described preferred embodiments, the active cooling module comprises a heat-conductive surface operable to conduct heat between the cooling module and tissues of a body.

According to still further features in the described preferred embodiments, the cryoprobe further comprises a flexible shaft attached to the treatment head, which may comprise flexibly attached rigid segments.

According to still further features in the described preferred embodiments, the cryoprobe further comprises a sensor operable to transmit data to a control module external to the cryoprobe. The sensor may be operable to transmit data over a wire, or by wireless transmission.

According to still further features in the described preferred embodiments, the sensor is a thermal sensor, or a pressure sensor.

According to still further features in the described preferred embodiments, the cryoprobe further comprises a plurality of sensors operable to transmit data to a control module external to the cryoprobe, and at least one of the plurality of sensors is a thermal sensor and at least one of the plurality of sensors is a pressure sensor.

According to another aspect of the present invention there is provided a shape-adaptable cryoprobe, having a treatment head operable to adaptively conform to a shape of an organic target, thereby enhancing transfer of heat between the treatment head and the organic target.

According to further features in preferred embodiments of the invention described below, the treatment head is operable to adaptively conform to a shape of a pulmonary vein ostium.

According to further features in preferred embodiments of the invention described below, the treatment head is inflatable, and operable to be cooled by Joule-Thomson cooling, and comprises a Joule-Thomson orifice.

According to further features in preferred embodiments of the invention described below, the treatment head is operable to be heated by Joule-Thomson heating.

According to further features in preferred embodiments of the invention described below, the treatment head comprises an expandable volume defined by a flexible inflatable external sleeve and is operable to be cooled by expanding cooling gas flowing into the expandable volume through a Joule-Thomson orifice.

According to further features in preferred embodiments of the invention described below, the treatment head comprises a Joule-Thomson cooler, a gas input lumen for supplying a pressurized cooling gas, a Joule-Thomson orifice at a termination of the gas input lumen, a flexible inflatable external sleeve operable to be inflated by gas passed through the Joule-Thomson orifice, a gas exhaust lumen for exhausting gas from the treatment head, and a gas exhaust valve operable to control flow of gas through the gas exhaust lumen.

According to further features in preferred embodiments of the invention described below, the cryoprobe further comprises an inner cooling module operable to be cooled by a Joule-Thomson cooler, and an exterior expansion volume defined within a flexible inflatable exterior sleeve, the exterior expansion volume being exterior to the inner cooling module. Preferably, the inner cooling module comprises a Joule-Thomson orifice, a fluid transfer lumen, a gas input lumen, and a gas exhaust lumen.

Preferably, the expansion volume is in fluid communication with the fluid transfer lumen and is operable to expand when filled by a fluid supplied under pressure through the fluid transfer lumen.

Preferably, the inner cooling module is operable to cool a fluid within the expansion volume.

According to still another aspect of the present invention there is provided a linear cryoprobe operable to apply cryogenic cooling to body tissues in an elongated pattern, which comprises:

a) a treatment head comprising a Joule-Thomson orifice and a heat-conducting surface so shaped that a ratio of length of the surface to width of the surface is greater than six to one;

b) a gas input lumen; and

c) a gas exhaust lumen;

According to further features in preferred embodiments of the invention described below, the treatment head further comprises an insulating shroud.

According to still another aspect of the present invention there is provided a system for treating cardiac arrhythmia, which comprises

a) a control module operable to receive data from a sensor;

b) a cryoprobe which comprises:

i) a treatment head comprises a Joule-Thomson orifice; and

ii) a gas input lumen operable to supply a pressurized gas to the Joule-Thomson orifice; and

b) a gas supply module operable to supply compressed gas to the gas input lumen.

According to further features in preferred embodiments of the invention described below, the cryoprobe further comprises a cryoprobe sensor operable to transmit data to the control module, preferably by wireless communication.

According to further features in preferred embodiments of the invention described below, the cryoprobe further comprises a plurality of cryoprobe sensors operable to transmit data to the control module, including thermal sensors and pressure sensors.

According to further features in preferred embodiments of the invention described below, the gas supply module comprises a plurality of sources of compressed gas.

According to further features in preferred embodiments of the invention described below, the plurality of sources comprises a source of compressed cooling gas.

According to further features in preferred embodiments of the invention described below, the plurality of sources comprises a source of compressed heating gas.

According to further features in preferred embodiments of the invention described below, the plurality of sources comprises a source of mixed cooling gas and heating gas.

According to further features in preferred embodiments of the invention described below, the plurality of sources comprises a plurality of sources of mixed cooling gas and heating gas.

According to further features in preferred embodiments of the invention described below, the system further comprises a cooling gas input valve controlling flow of cooling gas from the gas supply module into the gas input lumen.

According to further features in preferred embodiments of the invention described below, the cooling gas input valve is controllable by commands transmitted by the control module.

According to further features in preferred embodiments of the invention described below, the system further comprises a heating gas input valve controlling flow of heating gas from the gas supply module into the gas input lumen.

According to further features in preferred embodiments of the invention described below, the heating gas input valve is controllable by commands transmitted by the control module.

According to further features in preferred embodiments of the invention described below, the gas supply module comprises a heat exchanging configuration.

According to further features in preferred embodiments of the invention described below, the cryoprobe comprises a heat-exchanging configuration.

According to further features in preferred embodiments of the invention described below, the cryoprobe comprises a treatment head sized and shaped to fit a pulmonary vein ostium.

According to further features in preferred embodiments of the invention described below, the cryoprobe comprises a treatment head operable to adaptively conform to a shape of an organic target, thereby enhancing transfer of heat between the treatment head and the organic target.

According to further features in preferred embodiments of the invention described below, the cryoprobe is operable to adaptively conform to a shape of a pulmonary vein ostium.

According to further features in preferred embodiments of the invention described below, the treatment head is inflatable and comprises a Joule-Thomson orifice.

According to further features in preferred embodiments of the invention described below, the cryoprobe is operable to apply cryogenic cooling to body tissues in an elongated pattern.

According to further features in preferred embodiments of the invention described below, the cryoprobe comprises:

a) a treatment head which comprises a Joule-Thomson orifice and a heat-conducting surface so shaped that a ratio of length of the surface to width of the surface is greater than six to one;

b) a gas input lumen; and

c) a gas exhaust lumen.

According to still another aspect of the present invention there is provided a method for treating cardiac arrhythmia, which comprises:

a) introducing a cryoprobe into an atrium of a heart;

b) positioning the cryoprobe at an ostium of a pulmonary vein, in such a position that an active cooling module of the cryoprobe is in contact with tissues of the ostium;

c) cooling the active cooling module to a first temperature, the first temperature being such as to cause the cryoprobe to adhere to tissues of the ostium, thereby causing the cryoprobe to adhere to the tissues of the ostium;

d) testing the positioning of the cryoprobe by cooling the active cooling module to a second temperature, the second temperature being such as to create a temporary conduction block in the ostium if the cryoprobe is correctly positioned, thereby creating a temporary conduction block in the ostium if the cryoprobe is correctly positioned;

e) evaluating the positioning of the cryoprobe by determining whether the temporary conduction block was created by step (d);

f) if the temporary conductive block was created by step (d), cooling the active cooling module to a third temperature, the third temperature being such as to create a permanent conductive block in the ostium, thereby creating a permanent conductive block in the ostium, thereby treating the cardiac arrhythmia.

According to further features in preferred embodiments of the invention described below, the method further comprises

g) heating the cryoprobe to free the cryoprobe from the adhesion if a conductive block is not created by step (d); and

h) repositioning the cryoprobe at the ostium, and preferably

i) heating the cryoprobe after cooling the active cooling module to the third temperature, thereby releasing the cryoprobe from the adhesion after having created the conductive block.

According to further features in preferred embodiments of the invention described below, the cryoprobe is sized and formed to conform to a shape of a pulmonary vein ostium.

According to further features in preferred embodiments of the invention described below, the cryoprobe comprises an inflatable portion, and is operable to adaptively conform to a shape of a pulmonary vein ostium.

According to further features in preferred embodiments of the invention described below, the method further comprises

j) endoscopically introducing the cryoprobe into an atrium;

k) introducing a distal portion of the cryoprobe into an opening of a pulmonary vein; and

l) inflating the inflatable portion;

thereby adaptively conforming the cryoprobe a shape of the pulmonary vein ostium.

According to still another aspect of the present invention there is provided a method for treating cardiac arrhythmia, which comprises:

a) positioning at an exterior wall of a atrium a cryoprobe having a treatment head which comprises an elongated cooling surface;

b) cooling the cooling surface to a first temperature, the first temperature being such as to cause the cryoprobe to adhere to tissues of the atrium wall, thereby causing the cryoprobe to adhere to tissues of the atrium wall;

c) testing the positioning of the cryoprobe by cooling the cooling surface to a second temperature, the second temperature being such as to create a temporary conduction block in the atrium wall if the cryoprobe is correctly positioned, thereby creating a temporary conduction block in the atrium wall if the cryoprobe is correctly positioned;

d) evaluating the positioning of the cryoprobe by determining whether the temporary conduction block was created by step (d);

e) if the temporary conduction block was created by step (d), cooling the active cooling module to a third temperature, the third temperature being such as to create a permanent a permanent conduction block in the atrium wall, thereby creating a permanent conduction block in the atrium wall,

thereby treating the cardiac arrhythmia.

The present invention successfully addresses the shortcomings of the presently known configurations by providing a minimally invasive technique for creation of lesions capable of blocking pathological electrical conduction in atrial tissue, which technique permits treatment of atrial fibrillation and of other forms of cardiac arrhythmias, yet which does not require subjecting a patient to the trauma of open chest and open heart surgeries.

The present invention further successfully addresses the shortcomings of the presently known configurations by providing a therapeutic device and method enabling to place a therapeutic probe in or on a selected portion of a beating heart, and to maintain that probe accurately in place for a required duration of treatment, without resorting to heart immobilization.

The present invention still further successfully addresses the shortcomings of the presently known configurations by providing techniques for creating a circumferential conduction block in a pulmonary vein ostium, which techniques are minimally invasive, minimally traumatic, and which produce lesions sufficiently wide and deep to create a conduction block, yet which do not substantially disturb nor destroy the structural integrity of the atria.

The present invention still further successfully addresses the shortcomings of the presently known configurations by providing a device and method for mapping pathological areas responsible for arrhythmia, and for treating those pathological areas, in a single coordinated technique, while guaranteeing a high degree of reliability in ensuring that the problematic locations identified by mapping are indeed the locations subsequently subject to ablation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified schematic of a cryoprobe having a form-fitting treatment head adapted to conform to the shape of a pulmonary vein ostium, according to an embodiment of the present invention;

FIG. 2 is a simplified schematic presenting details of a Joule-Thomson cooler operable to cool a cooling module of a cryoprobe, according to an embodiment of the present invention;

FIG. 3 is a simplified schematic presenting currently preferred recommended dimensions for a treatment head of a cryoprobe, according to a preferred embodiment of the present invention;

FIG. 4 is a simplified schematic illustrating an alternate construction of a cooling module of a cryoprobe, according to an embodiment of the present invention;

FIG. 5 is a simplified schematic illustrating a further alternate construction of a cooling module of a cryoprobe, according to an embodiment of the present invention;

FIG. 6 is a simplified schematic presenting a configuration of a shaft of a cryoprobe, according to an embodiment of the present invention;

FIG. 7 is a simplified schematic presenting an alternate configuration of a shaft of a cryoprobe, according to an embodiment of the present invention;

FIG. 8 is a simplified schematic illustrating a shape-adaptable cryoprobe configured for endovascular insertion, according to an embodiment of the present invention;

FIG. 9 is a simplified schematic presenting a shape-adaptable cryoprobe configured for treating body tissues;

FIG. 10 is a simplified schematic illustrating a double-layered shape-adaptable cryoprobe configured for endoscopic insertion, according to an embodiment of the present invention;

FIG. 11 a simplified schematic illustrating a double-layered shape-adaptable cryoprobe configured for treatment of tissues, according to an embodiment of the present invention;

FIG. 12 is a simplified schematic illustrating a cryoprobe having an elongated treatment head, according to an embodiment of the present invention;

FIG. 13 is a simplified schematic of an elongated treatment head of a cryoprobe, according to an embodiment of the present invention;

FIG. 14 is a simplified schematic of a system for cryosurgery comprising a cryoprobe having a form-fitting treatment head, according to an embodiment of the present invention;

FIG. 15 is a simplified schematic of a system for cryosurgery comprising a shape-adaptable cryoprobe, according to an embodiment of the present invention;

FIG. 16 is a simplified schematic of a system for cryosurgery comprising a double-layered shape-adaptable cryoprobe, according to an embodiment of the present invention; and

FIG. 17 is a simplified schematic of a system for cryosurgery comprising a cryoprobe having an elongated head, according to an embodiment of the present invention.

### DESCRIPTION OF THE PREFERRED EMBODIMENTS

The present invention is of devices, systems, and methods for cryosurgical treatment of cardiac arrhythmia. Specifically, the present invention can be used to create a conduction block in a pulmonary vein ostium and in an atrial wall, to treat cardiac arrhythmia.

The principles and operation of cryoprobes specialized for treatment of atrial arrhythmia according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

To enhance clarity of the following descriptions, the following terms and phrases will first be defined:

The phrase “heat-exchanging configuration” is used herein to refer to component configurations traditionally known as “heat exchangers”, namely configurations of components situated in such a manner as to facilitate the passage of heat from one component to another. Examples of “heat-exchanging configurations” of components include a porous matrix used to facilitate heat exchange between components, a structure integrating a tunnel within a porous matrix, a structure including a coiled conduit within a porous matrix, a structure including a first conduit coiled around a second conduit, a structure including one conduit within another conduit, or any similar structure. It is to be noted that in the accompanying figures and in discussion of those figures hereinbelow, a particular exemplary configuration of a heat-exchanging configuration is shown in the figures, by way of illustration. It is to be understood that illustration of a particular configuration of heat-exchanging configuration in a figure is by way of example only, and is not intended to be limiting. The heat-exchanging configurations illustrated in the various figures may be any heat-exchanging configuration conforming to the definition of heat-exchanging configurations hereinabove.

The phrase “Joule-Thomson heat exchanger” as used herein refers, in general, to any device used for cryogenic cooling or for heating, in which a gas is passed from a first region of the device, wherein it is held under higher pressure, to a second region of the device, wherein it is enabled to expand to lower pressure. A Joule-Thomson heat exchanger may be a simple conduit, or it may include an orifice through which gas passes from the first, higher pressure, region of the device to the second, lower pressure, region of the device. A Joule-Thomson heat exchanger may further include a heat-exchanging configuration, for example a heat-exchanging configuration used to cool gasses within a first region of the device, prior to their expansion into a second region of the device.

The phrase “cooling gasses” is used herein to refer to gasses which have the property of becoming colder when passed through a Joule-Thomson heat exchanger. As is well known in the art, when gasses such as argon, nitrogen, air, krypton, CO₂, CF₄, xenon, and N₂O, and various other gasses pass from a region of higher pressure to a region of lower pressure in a Joule-Thomson heat exchanger, these gasses cool and may to some extent liquefy, creating a cryogenic pool of liquefied gas. This process cools the Joule-Thomson heat exchanger itself, and also cools any thermally conductive materials in contact therewith. A gas having the property of becoming colder when passing through a Joule-Thomson heat exchanger is referred to as a “cooling gas” in the following.

The phrase “heating gasses” is used herein to refer to gasses which have the property of becoming hotter when passed through a Joule-Thomson heat exchanger. Helium is an example of a gas having this property. When helium passes from a region of higher pressure to a region of lower pressure, it is heated as a result. Thus, passing helium through a Joule-Thomson heat exchanger has the effect of causing the helium to heat, thereby heating the Joule-Thomson heat exchanger itself and also heating any thermally conductive materials in contact therewith. Helium and other gasses having this property are referred to as “heating gasses” in the following.

As used herein, a “Joule-Thomson cooler” is a Joule-Thomson heat exchanger used for cooling. As used herein, “Joule Thomson cooling” is cooling by Joule Thomson cooler. As used herein, a “Joule-Thomson heater” is a Joule Thomson heat exchanger used for heating, and “Joule-Thomson heating” is heating by Joule-Thomson heater.

References hereinbelow to a pulmonary vein ostium are to be understood to refer to tissues within and immediately around a pulmonary vein ostium, that is, within and immediately around the point of entry of a pulmonary vein in an atrium of the heart. Thus, for example, reference to creation of a conduction block in a pulmonary vein ostium may be understood to include creation of a conduction block in epicardial tissue around and within a pulmonary vein ostium.

In discussion of the various figures described hereinbelow, like numbers refer to like parts.

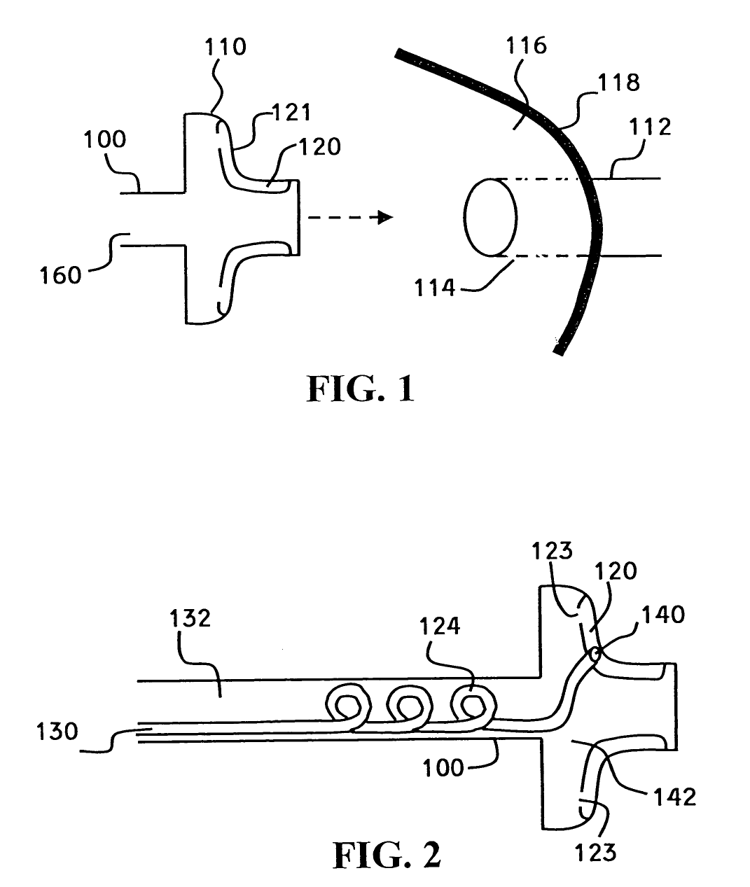

Referring now to the drawings, FIG. 1 is a is a simplified schematic of a cryoprobe having a form-fitting treatment head sized and formed to match the shape of a pulmonary vein ostium, according to an embodiment of the present invention.

FIG. 1 presents a cryoprobe 100 comprising a shaft 160 (shown here in abbreviated form) and a form-fitting treatment head 10 whose shape conforms to the shape of the ostium region 114 of a pulmonary vein 112, approached from within the left atrium 116 of a heart. Cryoprobe 100 is designed and constructed to treat atrial arrhythmia by use cryogenic cooling to create a circumferential conduction block in a pulmonary vein 112.

Cryoprobe 100 may be inserted into atrium 116 through open-heart surgery, yet in a preferred mode of operation cryoprobe 100 is inserted into atrium 116 in a minimally invasive procedure, and most preferably endovascularly.

Cryoprobe 100 comprises active cooling module 120, which in a preferred embodiment is formed as a circumferential zone on a distal face of treatment head 110, and is sized and formed to substantially conform to size and shape of ostium 114 of vein 112.

Cooling module 120 is operable to be cooled to cryoablation temperatures. Cooling module 120 preferably comprises a thermally conductive distal face 121, shaped and configured to form close contact with heart tissue at ostium 114, thereby enhancing heat transfer between cooling module 120 and tissues in and around ostium 114. Thus, cooling module 120 is operable to create a lesion, to damage or to ablate tissues of ostium region 114, and thereby to create a conduction block within region 114, without substantially disturbing the structural integrity of the atria.

Attention is now drawn to FIG. 2, which is a simplified schematic showing details of a Joule-Thomson cooler operable to cool cooling module 120, according to an embodiment of the present invention.

FIG. 2 presents a gas input lumen 130, operable to supply pressurized cooling gas to a Joule-Thomson orifice 140 situated in or near cooling module 120. Pressurized cooling gas from gas input lumen 130, passing through orifice 140, is enabled to expand. Expansion of pressurized cooling gas cools that gas, which consequently cools cooling module 120, and in particular distal face 121 of cooling module 120. If treatment head 110 of cryoprobe 100 is installed in close contact with tissues of ostium 114 and cooling module 120 is cooled by expansion of cooling gas from orifice 140, then thermal contact between tissues of ostium 114 and distal face 121 of cooling module 120 leads to cooling of those ostium tissues.

Expanded gasses are free to exit from cooling module 120 through one or more exits 123 in cooling module 120. Total cross-sectional area of exits 123 is significantly larger than that of orifice 140, thus substantially eliminating hydraulic resistance to gas outflow. Optional heat exchanging configuration 124 may be used to pre-cool cooling gas in gas input lumen 130, by exchanging beat between input gas in gas input lumen 130 and cold exhaust gas in gas exhaust lumen 132.

In a preferred embodiment of the present invention, gas input lumen 130 is further operable to supply pressurized heating gas to orifice 140. Expansion of pressurized heating gas heats that gas, which consequently heats cooling module 120, and in particular distal face 121 of cooling module 120. Optional heat exchanging configuration 124 can be used to pre-heat heating gas, by exchanging heat between hot expanded heating gas in gas exhaust lumen 132 and input heating gas in gas input lumen 130.

In a preferred mode of operation of cryoprobe 100, cooling of tissues of ostium 114 is used to produce several useful effects.

A first useful effect of cooling of tissues of ostium 114 by treatment head 110 is to cause treatment head 110 to adhere to those tissues. Such adherence is extremely useful, in that it creates a temporary bond between treatment head 110 and region 114, providing consistent positioning of treatment head 110 with respect to pulmonary vein 112, hence enabling a controlled and consistent process of further therapeutic cooling. This controlled and consistent process may be contrasted to processes of prior art arrhythmia therapies. Arrhythmia is preferably treated without stopping beating of the heart, yet the necessity of aiming a therapeutic probe at a moving target, and maintaining a contact with that target over an extended period of time while performing a therapeutic act, adds greatly to the difficulty of such therapeutic procedures. Adhesion, which occurs when cooling module 120 of treatment head 110 is cooled to a vicinity of −20° C., greatly simplifies continuation of a therapeutic procedure, because treatment head 110 maintains a consistent relationship to ostium 114, even though the heart is beating.

It is further noted that adhesion between treatment head 110 and tissues of ostium 114 is easily reversible. As described above, in a preferred embodiment gas input lumen 130 is operable to supply a heating gas to orifice 140. Supplying compressed heating gas to orifice 140 has an effect of heating treatment head 110, which liberates head 110 from adhesions caused by tissues freezing to head 110. Thus, it is possible for an operator to position head 110 with respect to a therapeutic target, cool head 110 sufficiently to cause adhesion, and inspect that positioning to determine if it is satisfactory. If so, the therapeutic process can continue. If not, head 110 is heated, the adhesion is released, and the operator is enabled to reposition head 110.

In an additional preferred mode of operation of cryoprobe 100, utilizing a second useful effect of cooling tissues of ostium 114, treatment head 110 is cooled to a moderate degree of cooling, preferably between −10° C. and −30° C, and most preferably between −15° C. and −25° C. Such moderate cooling causes a temporary blockage of electrical transmission through the cooled tissues. This temporary blockage is in effect a simulation of the permanent blockage that would be produced by more intense cooling. At a moderate cooling level, conduction blocking is temporary and reversible. Thus, in a preferred mode of operation, an operator is enabled to position head 110 at a therapeutic target, cool head 110 sufficiently to cause adhesion, and cool head 110 sufficiently to cause temporary blockage of electrical conductivity (generally, temporary conduction blockage takes place at temperatures similar to those which cause adhesion). The operator may then evaluate the results. If atrial arrhythmia is reduced or prevented, correct positioning of heat 110 is confirmed. If, on the other hand, arrhythmia is not significantly corrected, then no permanent damage has been done to the cooled tissues, head 110 is heated to release adhesion, and head 10 may be repositioned.

Positioning, adhering, testing, freeing, and repositioning may be repeated until a position is found which successfully reduces arrhythmia when tested by moderate cooling.

In an additional preferred mode of operation of cryoprobe 100, utilizing a third useful effect of cooling tissues of ostium 114, once appropriate positioning of head 110 has been achieved and tested, ostial tissues 114 are further cooled, to effect permanent blockage of electrical conductivity.

To permanently affect blockage of electrical conductivity in the treated tissues, cooling module 120 is preferably cooled to a temperature between −30° C. and −120° C., and more preferably between −40° C. and −80° C., to create permanent electrical conductivity blockage.

Heating of head 110 may subsequently optionally be practiced, to secure release of adhesions between head 110 and tissues which adhered to head 110 when frozen.

Attention is now drawn to FIG. 3, which is a simplified schematic presenting currently preferred dimensions for treatment head 110, according to a preferred embodiment of the present invention. Diameter 170 is preferably between 5 mm and 25 mm, and most preferably between 10 mm and 20 mm. Diameter 171 is preferably between 10 mm and 35 mm, most preferably between 15 mm and 25 mm. Distance 172 is preferably between 5 mm and 30 mm, and most preferably between 10 mm and 20 mm. In a preferred mode of utilization, a surgeon would be supplied with a plurality of cryoprobes 100 of varying dimension, and would thus be enabled to choose an appropriate model, in view of the actual size of a patient's ostium, after access is made and the ostium observed.

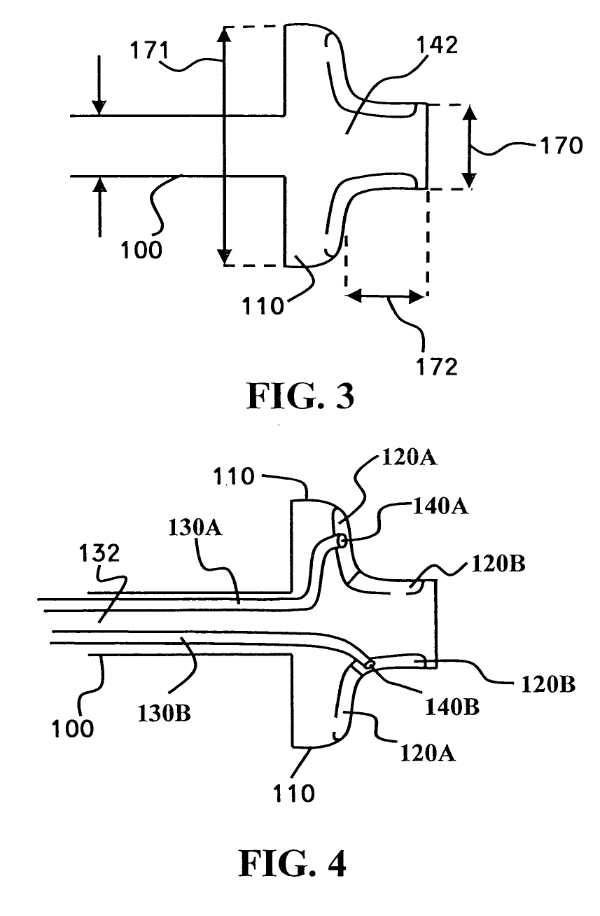

Attention is now drawn to FIG. 4, which is a simplified schematic illustrating an alternate construction of cooling module 120, according to an embodiment of the present invention. FIG. 4 presents a treatment head 110 having a plurality of separately coolable cooling modules 120, concentrically arranged. Exemplary modules are designated in FIG. 4 as 120A and 120B. Cooling module 120A receives gas from a gas input lumen 130A. Cooling module 120B receives gas from gas input lumen 130B. Flow of gas in each of gas input lumens 130A and 130B is individually controllable, consequently cooling of cooling modules 120A and 120B is individually controllable as well.

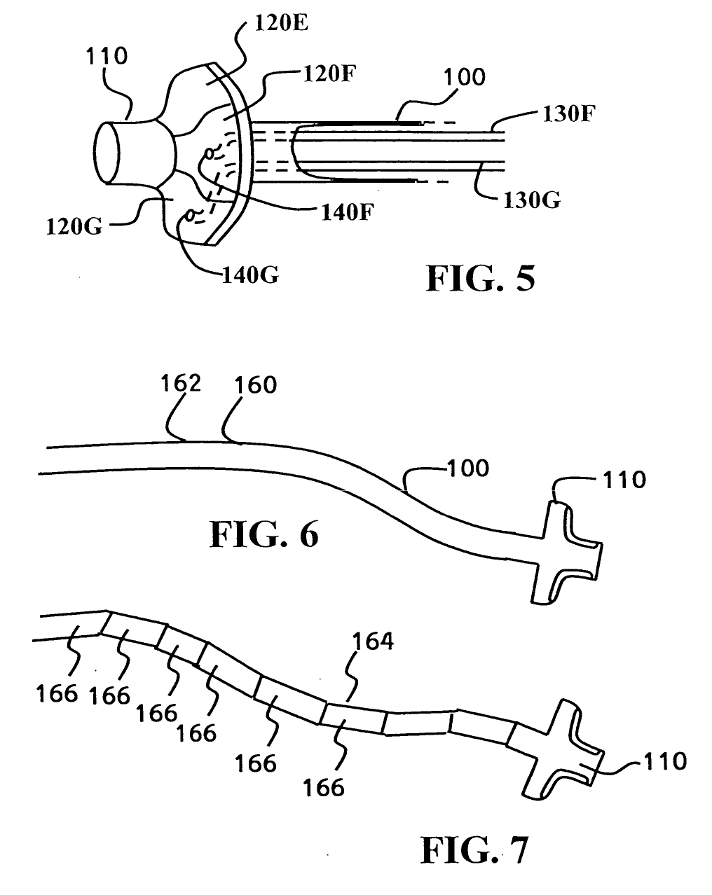

Attention is now drawn to FIG. 5, which is a simplified schematic illustrating a further alternate construction of cooling module 120, according to an embodiment of the present invention. FIG. 5 presents a treatment head 110 having a plurality of separately coolable cooling modules 120, radially arranged. Exemplary modules are designated in FIG. 5 as 120E, 120F, and 120G. Cooling module 120F receives gas from a gas input lumen 130F. Cooling module 120G receives gas from gas input lumen 130G. Other cooling modules 120 are similarly supplied with gas (additional gas input lumens not shown). Flow of gas in each gas input lumen (e.g., 130F and 130G) is individually controllable, consequently cooling of each cooling module 120 is individually controllable as well.

Attention is now drawn to FIG. 6, which is a simplified schematic presenting a configuration of shaft 160 of cryoprobe 100, according to an embodiment of the present invention. Shaft 160 of probe 100 is a continuously flexible shaft 162, preferably constructed of a flexible material, such as, for example, Biocompatible Tygon(R).

Attention is now drawn to FIG. 7, which is a simplified schematic presenting an alternate configuration of shaft 160 of cryoprobe 100, according to an embodiment of the present invention. In this alternative configuration, shaft 160 of probe 100 is a modularly flexible shaft 164, comprising a plurality of rigid segments 166, flexibly connected to each other.

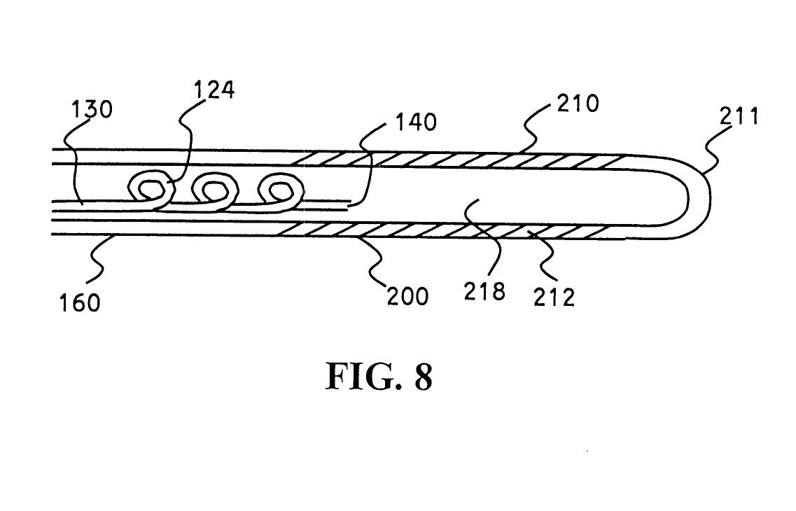

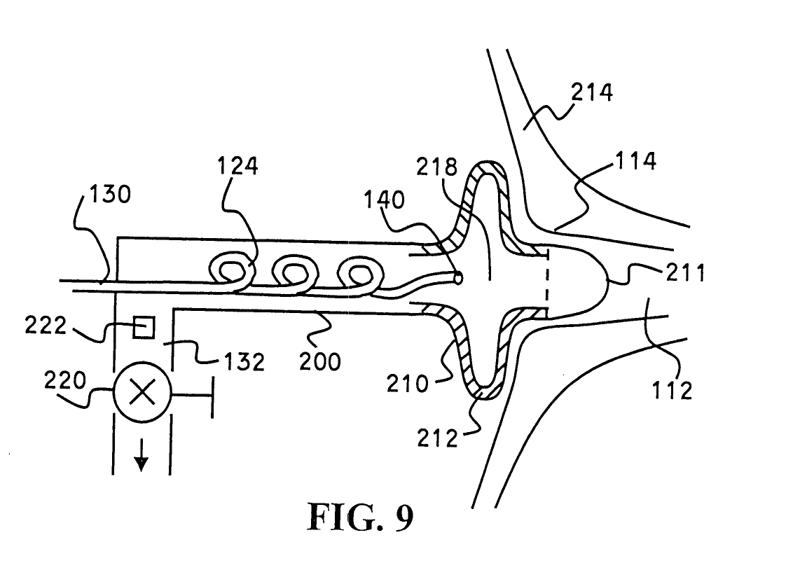

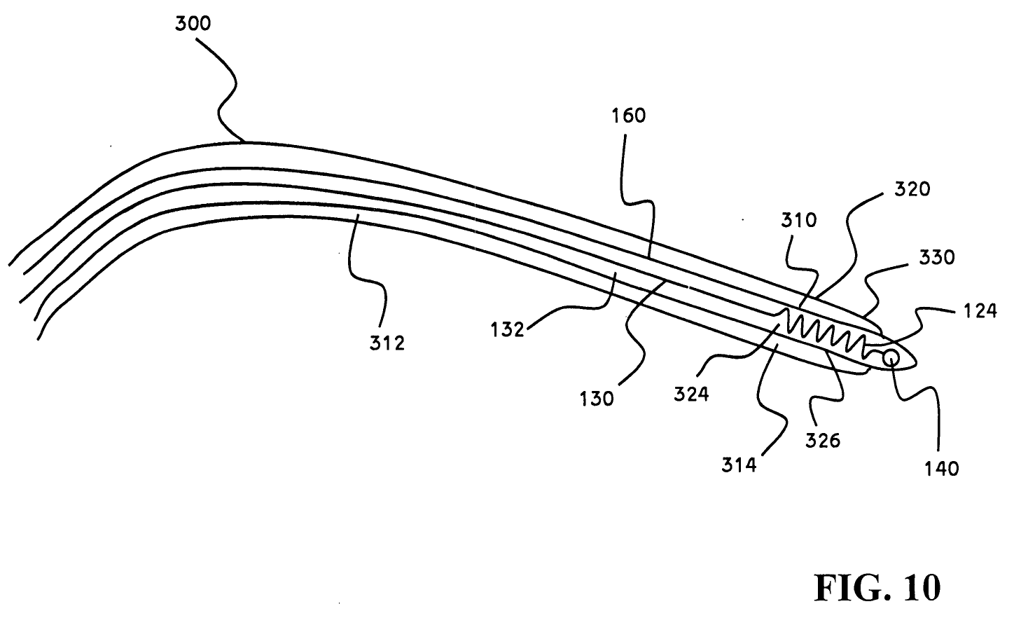

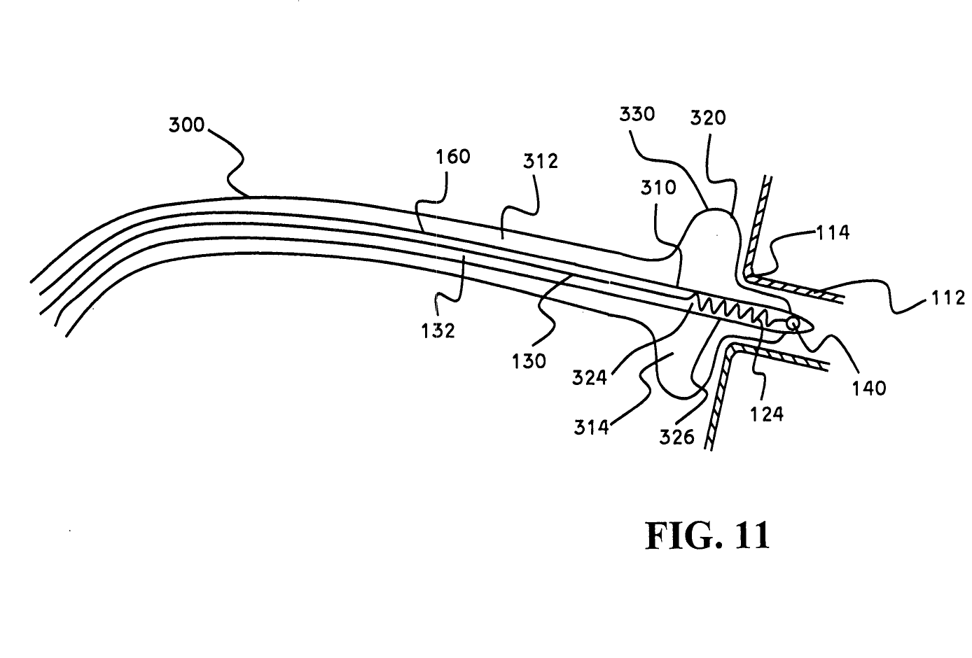

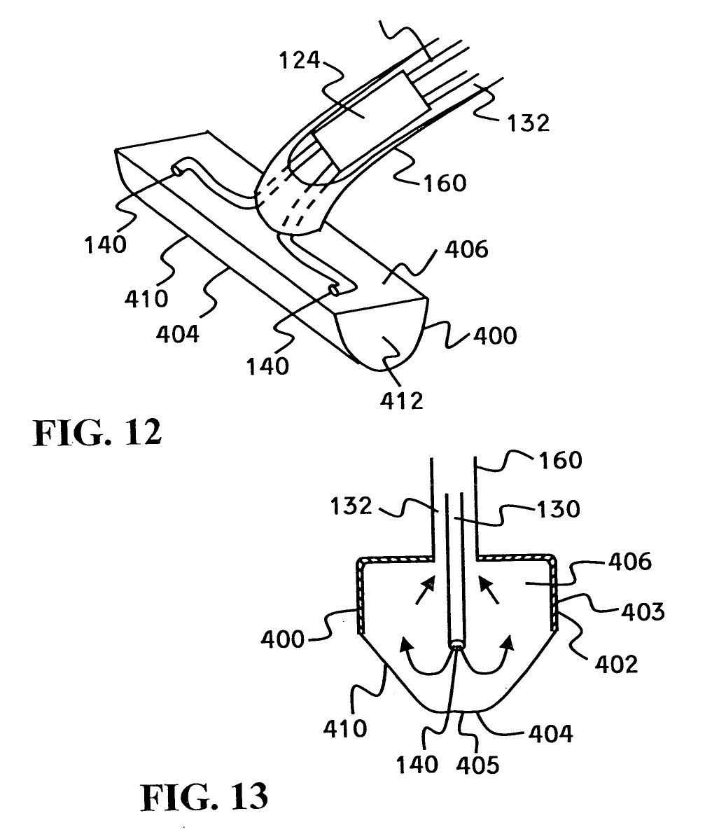

It is noted that flexible shaft 162, illustrated in FIG. 6, and modularly flexible shaft 164, illustrated in FIG. 7, are optional implementations of shaft 160 of cryoprobe 100, described hereinabove with reference to FIGS. 1-5. It is further noted that flexible shaft 162, illustrated in FIG. 6, and modularly flexible shaft 164, illustrated in FIG. 7, are optional implementations of shaft 160 of cryoprobe 200, described hereinbelow with reference to FIGS. 8-9, and of shaft 160 of cryoprobe 300, described hereinbelow with reference to FIGS. 10-11, and of cryoprobe 400, described hereinbelow with reference to FIG. 12.

Attention is now drawn to FIG. 8, which is a simplified schematic illustrating a shape-adaptable cryoprobe 200 configured for endovascular insertion, according to an embodiment of the present invention.

Shape-adaptable cryoprobe 200 comprises an inflatable/deflatable head 210 having an expandable internal volume 218 hermetically contained within a flexible inflatable external sleeve 212. When deflated, cryoprobe 200 is configured for endovascular insertion or for other uses requiring passage through narrow openings. When deflated, diameter of head 210 is preferably not substantially larger than diameter of shaft 160.

Attention is now drawn to FIG. 9, which is a simplified schematic presenting shape-adaptable cryoprobe 200 configured for treating ostial tissues 114, or other tissues. In operation, cooling gas supplied through gas input lumen 130, and passing through an optional heat-exchanging configuration 124, expands though Joule-Thomson orifice 140 into internal volume 218. Cooling gas passing through orifice 140 has a double role. First, expanded cooling gas is cold, and serves to cool flexible inflatable external sleeve 212 of inflatable/deflatable head 210. Second, gas expanding into external sleeve 212 inflates sleeve 212, expanding head 210 into a form which may bring it into close contact with tissues to be treated.

In a recommended method of use, with head 210 deflated, distal portion 211 of inflatable/deflatable head 210 is first inserted into the opening of pulmonary vein 112. Inflatable/deflatable head 210 is then both cooled and inflated by cooling gas or by a mixture of gasses, causing it to expand against ostial tissues 114 and neighboring tissues. Ostial tissues 114 and optionally other neighboring tissues 214 may then be treated with various degrees of cryogenic cooling, as described hereinabove.

Internal volume 218 communicates with gas exhaust lumen 132, whereby expanded gas is eliminated from cryoprobe 200.

According to a preferred embodiment, a desired pressure is maintained in volume 218 by appropriate use of a gas exhaust valve 220 controlling outflow of gas from gas output lumen 132. Gas exhaust valve 220 is optionally implemented as a remotely-controlled valve responsive to commands received from a command module 450 (not shown in FIG. 9). In a preferred embodiment command module 450 is operable to receive pressure data from a pressure sensor 222, which measures pressure in gas exhaust lumen 132 and communicates its measurements to command module 450, either by wire or by wireless communication.

In a preferred embodiment, gas input lumen 130 is operable to receive heating gas as well as cooling gas, and further operable to receive a mixture of heating and cooling gasses. Pressure can thus be introduced into volume 218 using an expanded gas which cools head 210, or using an expanded gas which heats head 210, or using an expanded gas which leaves temperature of head 210 substantially unchanged.

In a recommended use, once head 210 has been positioned and inflated as described hereinabove, cryoprobe 200 is useable in the various ways, and with the various effects, as were described hereinabove with reference to uses of cooling and heating of cryoprobe 100, particularly with reference to the discussion of FIG. 2.

Attention is now drawn to FIG. 10, which is a simplified schematic illustrating a double-layered shape-adaptable cryoprobe 300 configured for endoscopic insertion, according to an embodiment of the present invention.

Cryoprobe 300 shares many of the features, uses, and advantages of cryoprobe 200 illustrated by FIG. 8 and FIG. 9, yet cryoprobe 300 is differently constructed. Cryoprobe 300 comprises a shaft 160 and a shape-adaptable treatment head 330.

Shaft 160 comprises an input gas lumen 130, a gas exhaust lumen 132, and a fluid transfer lumen 312.

Treatment head 330 comprises a flexible inflatable exterior sleeve 320, an inner cooler 310 (also called an inner cooling module 310), and an exterior expansion volume 314 defined within exterior sleeve 320 and exterior to inner cooler 310. Exterior volume 314 is hermetically contained by sleeve 320.

Inner cooler 310 is formed within a cooler wall 326, which defines and hermetically contains a cooler interior volume 324. Inner cooler 310 further comprises a Joule-Thomson orifice 140 through which pressurized gas from gas input lumen 130 may expand into interior volume 324. As explained hereinabove, cooling gas expanding through orifice 140 will cool inner cooler 310, and heating gas expanding through orifice 140 will heat inner cooler 310. Expanded gas exhausts from volume 324 through gas exhaust lumen 132.

When cryoprobe 300 is in a deflated configuration, as shown in FIG. 10, exterior expansion volume 314 is preferentially substantially empty of fluid.

Exterior expansion volume 314 is in fluid communication with fluid transmission lumen 312 extending through shaft 160. Fluid transmission lumen 312 is operable to transfer a fluid into and out of exterior volume 314.

To deflate treatment head 330, fluid is drained or allowed to drain from exterior volume 314, through fluid transmission lumen 312, thereby emptying or partially emptying exterior volume 312 and deflating exterior sleeve 320, thereby contracting head 330. In a preferred embodiment, diameter of treatment head 330 when contracted is not substantially larger than diameter of shaft 160, thereby facilitating insertion of cryoprobe 300 through narrow openings, and in particular facilitating endovascular introduction and deployment of probe 300.

Attention is now drawn to FIG. 11, which is a simplified schematic presenting cryoprobe 300 in inflated configuration.

To inflate treatment head 330, a fluid 316 is forced under pressure through fluid transmission lumen 312 into exterior expansion volume 314, thereby inflating exterior sleeve 320 and expanding treatment head 330, as illustrated by FIG. 11. In a preferred embodiment, fluid 316 is a liquid, yet fluid 316 may be a gas.

When it is desired to cool treatment head 330, cooling gas is supplied under pressure, through gas input lumen 130, to Joule-Thomson orifice 140, whence it expands into interior volume 324, is cooled by expansion, and cools cooler wall 326. Cooler wall 326 is preferably constructed of heat-transmissive material, such as a metal, to facilitate transfer of heat between inner cooler 310 and fluid 316. Thus, cooling cooler wall 326 cools fluid 316, which in turn cools exterior sleeve 320. Thus, cooling inner cooler 310 cools exterior sleeve 320.

In use, exterior sleeve 320 is positioned in contact or near proximity with tissues of ostium region 114 which is desired to treat, and cooling inner cooler 310 when head 330 is positioned in contact with, or close to, tissues of ostium region 114 cools those tissues.

Recommended uses of cryoprobe 300 include positioning and inflating cryoprobe 300 as described hereinabove, and then cooling and heating cryoprobe 300 to various temperatures, to affect ostial tissues 114, as discussed hereinabove with respect to cryoprobe 100, particularly with reference to the discussion of FIG. 2.

As shown in FIGS. 10 and 11, cryoprobe 300 optionally comprises one or more heat exchanging configurations, similar to that described hereinabove with reference to cryoprobe 100, for pre-cooling cooling gas and for pre-heating heating gas directed through gas input lumen 130 into cooler 310.

Attention is now drawn to FIG. 12, which is a simplified schematic illustrating a cryoprobe having an elongated head, according to an embodiment of the present invention.

A well-known method of treatment of atrial arrhythmia comprises practicing long and narrow lesions in exterior portions of an atrial wall. FIG. 12 presents a cryoprobe 400 adapted to producing such lesions.

Cryoprobe 400 comprises an elongated treatment head 410 and a shaft 160.

Shaft 160 comprises a gas input lumen 130, a gas exhaust lumen 132, and one or more optional heat exchanging configurations 124.

Treatment head 410 comprises at least one and preferably a plurality of Joule-Thomson orifices, through which compressed cooling gas and compressed heating gas from gas input lumen 132 passes into an expansion chamber 406. Cooling gas, expanding into chamber 406 and cooled by expansion, cools expansion chamber 406.

Treatment head 410 has an elongated shape, that is, treatment head 410 is relatively longer than it is wide. A preferred ration of length to width is preferably greater than 6 to 1. For example, a recommended dimension for a preferred embodiment of treatment head 410 is of a length between 10 mm and 80 mm, and a width of between 1 mm and 10 mm. It is noted, however, that in a preferred mode of utilization, a surgeon would be supplied with a plurality of cryoprobes 400 of varying dimension, and would thus be enabled to choose an appropriate model, in view of the actual size of a treatment locus, once access is made and the locus observed.

Attention is now drawn to FIG. 13, which is a simplified schematic of treatment head 410 of cryoprobe 400, according to an embodiment of the present invention. FIG. 13 illustrates treatment head 410 as viewed from a narrow side. That is, FIG. 13 illustrates treatment head 410 as viewed from the side designated 412 in FIG. 12.

In FIG. 13, arrows illustrate passage of a gas (e.g., a cooling gas) from gas input lumen 130, expanding into expansion chamber 406, from whence gas is exhausted through gas exhaust lumen 132. Expansion of cooling gas into chamber 406 cools chamber 406. An insulating shroud 402, preferably of biomedical plastic material such as Teflon®, provides insulation on an exterior wall of proximal portion 403 of head 410, and serves to protect tissues in contact with proximal portion 403 from being unduly cooled by contact with treatment head 410. A thermally conductive surface 404, for example a metal strip, is provided on distal portion 405 of head 110, and serves to enhance thermal conductivity between head 410 and body tissues. Thus, when treatment head 410 is cooled, tissues touching conductive strip 404 or in close proximity to conductive strip 404 will be efficiently cooled by head 410, whereas tissues touching or in close proximity to proximal portion 403 of head 410 will be protected by insulating shroud 402 and will be relatively uninfluenced by treatment head 410.

In a recommended usage, treatment head 410 of cryoprobe 400 is positioned against, and in contact with, an exterior surface of an atrial wall, where treatment head 410 is cooled to create a conduction block within atrial wall tissues. Recommended usages for cryoprobe 400 include those outlined above with respect to cryoprobe 100 and in particular with reference to FIG. 2.

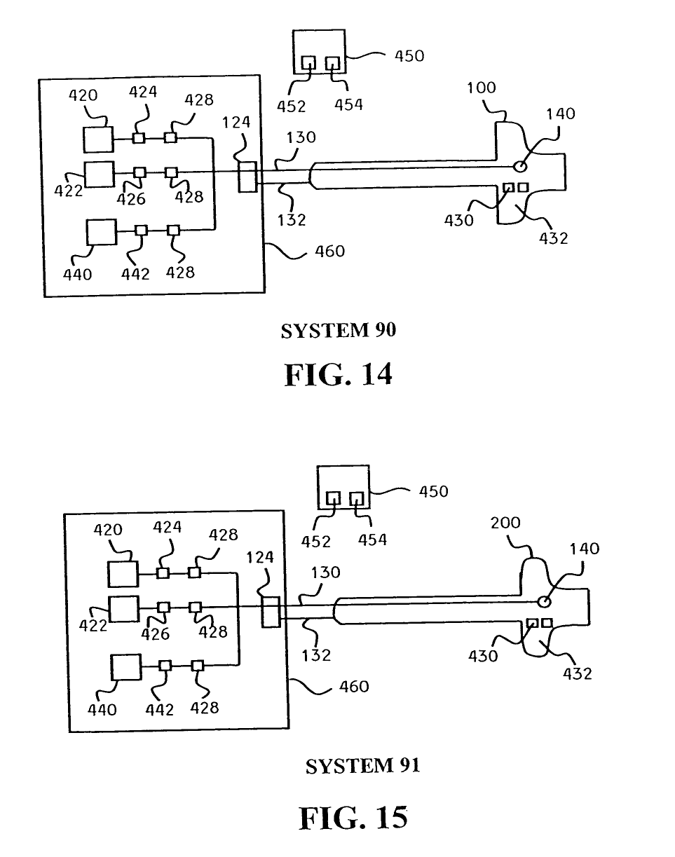

Attention is now drawn to FIG. 14, which is a simplified schematic of a system for cryosurgery comprising a cryoprobe having a form-fitting treatment head, according to a embodiment of the present invention.

System 90, illustrated by FIG. 14, is particularly recommended for treating of atrial arrhythmia, and in particular for forming a conduction block in a pulmonary vein ostium.

System 90 comprises a cryoprobe 100, as described hereinabove with particular reference to FIGS. 1-5. System 90 further comprises a gas supply module 460 and a command module 450.

Gas supply module 460 is operable to supply compressed gas to gas input lumen 130 of cryoprobe 100.

Gas supply module 460 comprises a cooling gas source 420, which is a source of compressed cooling gas, and a heating gas source 422, which is a source of compressed heating gas. Flow of gas from cooling gas source 420 is controlled by cooling gas input valve 424, which is preferably a remotely controllable valve. Flow of gas from heating gas source 422 is controlled by heating gas input valve 426, which is preferably a remotely controllable valve. Gas supply module 450 further comprises one-way valves 428.

Gas supply module 460 optionally further comprises a mixed gas source 440, which is a source of a mixture of cooling gas and heating gas in selected proportion. Flow of gas from mixed gas source 440 is controlled by mixed gas input valve 442, which is preferably a remotely controllable valve.

Gas supply module 460 further optionally comprises a heat-exchanging configuration 124, operable to pre-cool cooling gas flowing towards gas input lumen 130 by transferring heat from cooling gas flowing towards gas input lumen 130 to cold cooling gas exhausting from gas exhaust lumen 132.

Heat exchanging configuration 124 is further operable to pre-heat heating gas by transferring heat from hot heating gas exhausting from gas exhaust lumen 132, which has been heated by expansion, to compressed heating gas flowing towards gas input lumen 130.

Gas supply module 460 may further comprise other optional means for cooling of cooling gas flowing towards gas input lumen 130, and for heating of heating gas flowing towards gas input lumen 130.

Command module 450 is operable to receive real-time data from one or more optional thermal sensors 430 and one or more optional pressure sensors 432. Thermal sensor 430 may be a thermocouple, or other form of heat sensor.

Thermal sensors 430 and pressure sensors 432 may be situated within treatment head 110 of cryoprobe 100, as illustrated in FIG. 14, or alternatively maybe be situated in shaft 160 of cryoprobe 100, or further alternatively may be situated at various points within gas supply module 450.

Thermal sensors 430 are operable to communicate temperature data to command module 450 in real time. Pressure sensors 432 are also operable to communicate temperature data to command module 450 in real time.

Command module 450 is operable to receive data from thermal sensors 430 and from pressure sensors 432. Command module 450 is further operable to receive instructions from an operator. Command module 450 preferably comprises a memory 452 and a display 454. Command module 450 is preferably operable to display data received from sensors 430 and 432, and to display instructions received from an operator. Command module 450 is operable to send commands to cooling gas input valve 424, to heating gas input valve 426, and to mixed gas input valve 442, and is optionally further operable to send commands to other valves and controls of system 90.

Command module 450 is further preferably operable to algorithmically select or generate commands to be sent to gas input valve 424 and to heating gas input valve 426 and to mixed gas input valve 442, such commands being based on algorithmic evaluations of data received from sensors 430 and 432, and further based on instructions received from an operator. Algorithms thus used may be stored in memory 452.

Command module 450 is further operable to record in memory 452, for later display and analysis, data received from sensors 430 and 432 and instructions received from an operator.

In a preferred use, command module 450 is operable to respond to instructions from an operator by adjusting flow from a plurality of gas sources, to produce a mixture which, when expanded in a Joule-Thomson orifice, will produce a selected degree of cooling. As was noted hereinabove, selected steps in a therapeutic process of treatment of atrial arrhythmia may require selected degrees of cooling during different phases of a treatment process. Command module 450 is preferably operable to deliver to gas input lumen 130 a selected mixture of gas such as will produce a selected degree of cooling in treatment head 110. In a preferred embodiment, command module 450 is operable to deliver this selected mixture of gas according to a pre-selected mixture of cooling gas and of heating gas. In a further preferred embodiment, command module 450 is operable to deliver this selected mixture of gas according to algorithmically selected commands to gas input valves 424, 426, and 442, in response to temperature and pressure data receive from sensors 430 and 432.

An alternate preferred embodiment of gas supply module 460 (not shown) presents a plurality of mixed gas sources 440, (e.g., 440A, 440B, etc.), each operable to supply a mixture of heating gas and cooling gas in a selected proportion. Preferably, each of mixed gas sources 440 presents a mixture operable to supply a desired degree of cooling for a particular phase of treatment of arrhythmia, as described hereinabove.

In an optional embodiment of system 90, wherein cryoprobe 100 comprises a plurality of gas input lumens, gas supply module 460 optionally comprises a plurality of cooling gas input valves 424 (e.g., 424A, 424B, 424C), a plurality of heating gas input valves 426 (e.g., 426A, 426B, 426C), and optionally a plurality of mixed gas input valves (e.g., 442A, 442B, 442C), (not shown in FIG. 14). In a preferred embodiment, command module 450 is operable to control each of said plurality of gas input values individually, thereby individually controlling cooling and heating of each of a plurality of active cooling modules 120 (e.g., 120A, 120B, 120E, 120F, 120G).

Attention is now drawn to FIG. 15, which is a simplified schematic of a system for cryosurgery comprising a shape-adaptable cryoprobe, according to an embodiment of the present invention.

System 91, illustrated by FIG. 15, is particularly recommended for treating atrial arrhythmia, and in particular for forming a conduction block in a pulmonary vein ostium.

System 91 comprises a shape-adaptable cryoprobe 200, as described hereinabove with particular reference to FIG. 8 and FIG. 9. System 90 further comprises a gas supply module 460 and a command module 450.

Gas supply module 460 is operable to supply compressed gas to gas input lumen 130 of cryoprobe 200.

Gas supply module 460 comprises a cooling gas source 420, which is a source of compressed cooling gas, and a heating gas source 422, which is a source of compressed heating gas. Flow of gas from cooling gas source 420 is controlled by cooling gas input valve 424, which is preferably a remotely controllable valve. Flow of gas from heating gas source 422 is controlled by heating gas input valve 426, which is preferably a remotely controllable valve. Gas supply module 450 further comprises one-way valves 428.

Gas supply module 460 optionally further comprises a mixed gas source 440, which is a source of a mixture of cooling gas and heating gas in selected proportion. Flow of gas from mixed gas source 440 is controlled by mixed gas input valve 442, which is preferably a remotely controllable valve.

Gas supply module 460 further optionally comprises a heat-exchanging configuration 124, operable to pre-cool cooling gas flowing towards gas input lumen 130 by transferring heat from cooling gas flowing towards gas input lumen 130 to cold cooling gas exhausting from gas exhaust lumen 132.

Heat exchanging configuration 124 is further operable to pre-heat heating gas by transferring heat from hot heating gas exhausting from gas exhaust lumen 132, which has been heated by expansion, to compressed heating gas flowing towards gas input lumen 130.

Gas supply module 460 may further comprise other optional means to cool cooling gas flowing towards gas input lumen 130, and to heat heating gas flowing towards gas input lumen 130.

Command module 450 is operable to receive real-time data from one or more optional thermal sensors 430 and one or more optional pressure sensors 432. Thermal sensor 430 may be a thermocouple, or other form of heat sensor.

Thermal sensors 430 and pressure sensors 432 may be situated within treatment head 210 of cryoprobe 200, as illustrated in FIG. 15, or alternatively maybe be situated in shaft 160 of cryoprobe 200, or further alternatively may be situated at various points within gas supply module 450.

Thermal sensors 430 are operable to communicate temperature data to command module 450 in real time. Pressure sensors 432 are also operable to communicate temperature data to command module 450 in real time.

Command module 450 is operable to receive data from thermal sensors 430 and from pressure sensors 432. Command module 450 is further operable to receive instructions from an operator. Command module 450 preferably comprises a memory 452 and a display 454. Command module 450 is preferably operable to display data received from sensors 430 and 432, and to display instructions received from an operator. Command module 450 is operable to send commands to cooling gas input valve 424, to heating gas input valve 426, and to mixed gas input valve 442, and is optionally further operable to send commands to other valves and controls of system 91.

Command module 450 is further preferably operable to algorithmically select or generate commands to be sent to gas input valve 424, to heating gas input valve 426, and to mixed gas input valve 442, such commands being based on algorithmic evaluations of data received from sensors 430 and 432, and further based on instructions received from an operator. Algorithms thus used may be stored in memory 452.

Command module 450 is further operable to record in memory 452, for later display and analysis, data received from sensors 430 and 432 and instructions received from an operator.

It is further noted that in system 91, command module 450 is operable to send commands to gas exhaust valve 220, and thus to control outflow of gas from gas output lumen 132. Thus, by coordinating inflow of gas from gas supply module 460 into gas input lumen 130, and outflow of gas from gas output lumen 132, command module 450 is operable to control pressure within internal volume 218 of head 210 of cryoprobe 200, and thereby to control inflation and deflation of inflatable/deflatable head 210 of cryoprobe 200. Control module 450 preferably controls inflation and deflation of head 210 under algorithmic control, according to pre-set programmed instructions, or according to instructions received from an operator in real time.

In a preferred use, command module 450 is operable to respond to instructions from an operator by adjusting flow from a plurality of gas sources, to produce a mixture which, when expanded in a Joule-Thomson orifice, will produce a selected degree of cooling. As was noted hereinabove, selected steps in a therapeutic process of treatment of atrial arrhythmia may require selected degrees of cooling during different phases of a treatment process. Command module 450 is preferably operable to deliver to gas input lumen 130 a selected mixture of gas such as will produce a selected degree of cooling in treatment head 210. In a preferred embodiment, command module 450 is operable to deliver this selected mixture of gas according to a pre-selected mixture of cooling gas and of heating gas. In a further preferred embodiment, command module 450 is operable to deliver this selected mixture of gas according to algorithmically selected commands to gas input valves 424, 426, and 442, in response to temperature and pressure data receive from sensors 430 and 432.

An alternate preferred embodiment of gas supply module 460 (not shown) presents a plurality of mixed gas sources 440, (e.g., 440A, 440B, etc.), each operable to supply a mixture of heating gas and cooling gas in a selected proportion. Preferably, each of mixed gas sources 440 presents a mixture operable to supply a desired degree of cooling for a particular phase of treatment of arrhythmia, as described hereinabove.

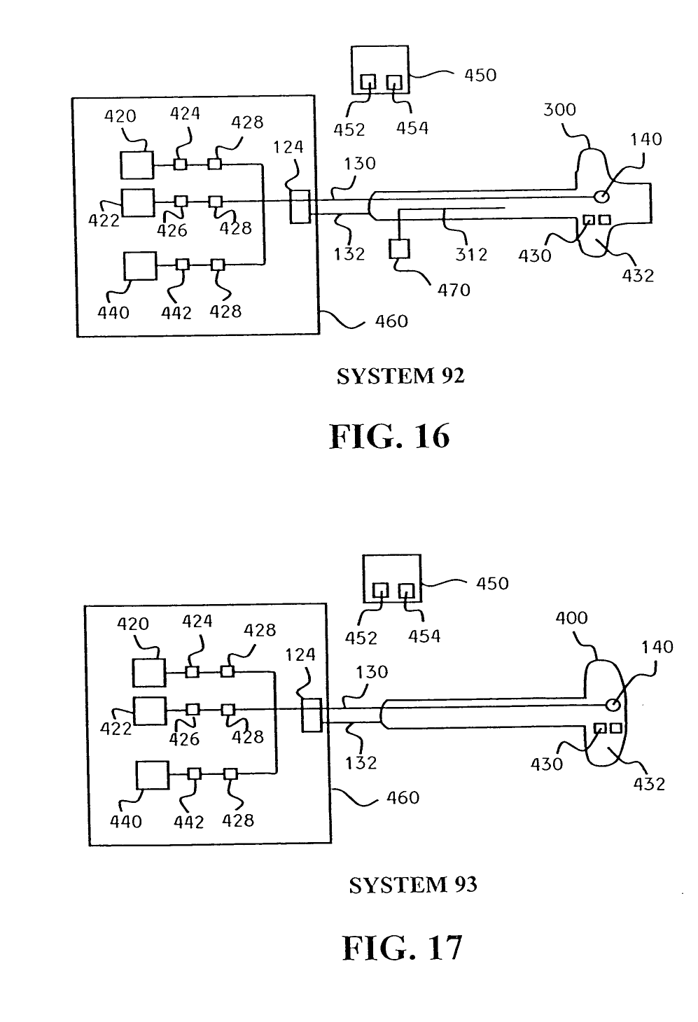

Attention is now drawn to FIG. 16, which is a simplified schematic of a system for cryosurgery comprising a double-layered shape-adaptable cryoprobe, according to a embodiment of the present invention.

System 92, illustrated by FIG. 16, is particularly recommended for treating atrial arrhythmia, and in particular for forming a conduction block in a pulmonary vein ostium.

System 92 comprises a double-layered shape-adaptable cryoprobe 300, as described hereinabove with particular reference to FIG. 10 and FIG. 11. System 92 further comprises a gas supply module 460, a command module 450, and a fluid pump 470.

Fluid pump 470 is operable to pump fluid into fluid transfer lumen 312 of cryoprobe 300. Fluid pump 470 is preferable also operable to pump fluid out of fluid transfer lumen 312, yet alternatively fluid pump 470 may be operable to allow fluid to drain from fluid transfer lumen 312. Fluid pump 470 is preferably operable to respond to commands from command module 450.

Gas supply module 460 is operable to supply compressed gas to gas input lumen 130 of cryoprobe 300.

Gas supply module 460 comprises a cooling gas source 420, which is a source of compressed cooling gas, and a heating gas source 422, which is a source of compressed heating gas. Flow of gas from cooling gas source 420 is controlled by cooling gas input valve 424, which is preferably a remotely controllable valve. Flow of gas from heating gas source 422 is controlled by heating gas input valve 426, which is preferably a remotely controllable valve. Gas supply module 450 further comprises one-way valves 428.

Gas supply module 460 optionally further comprises a mixed gas source 440, which is a source of a mixture of cooling gas and heating gas in selected proportion. Flow of gas from mixed gas source 440 is controlled by heating gas input valve 426, which is preferably a remotely controllable valve.

Gas supply module 460 further optionally comprises a heat-exchanging configuration 124, operable to pre-cool cooling gas flowing towards gas input lumen 130 by transferring heat from cooling gas flowing towards gas input lumen 130 to cold cooling gas exhausting from gas exhaust lumen 132.

Heat exchanging configuration 124 is further operable to pre-heat heating gas by transferring heat from hot heating gas exhausting from gas exhaust lumen 132, which has been heated by expansion, to compressed heating gas flowing towards gas input lumen 130.

Gas supply module 460 may further comprise other optional means to cool cooling gas flowing towards gas input lumen 130, and to heat heating gas flowing towards gas input lumen 130.

Command module 450 is operable to receive real-time data from one or more optional thermal sensors 430 and one or more optional pressure sensors 432. Thermal sensor 430 may be a thermocouple, or other form of heat sensor.

Thermal sensors 430 and pressure sensors 432 may be situated within treatment head 330 of cryoprobe 300, as illustrated in FIG. 16, or alternatively maybe be situated in shaft 160 of cryoprobe 300, or further alternatively may be situated at various points within gas supply module 450.

Thermal sensors 430 are operable to communicate temperature data to command module 450 in real time. Pressure sensors 432 are also operable to communicate temperature data to command module 450 in real time.

Command module 450 is operable to receive data from thermal sensors 430 and from pressure sensors 432. Command module 450 is further operable to receive instructions from an operator. Command module 450 preferably comprises a memory 452 and a display 454. Command module 450 is preferably operable to display data received from sensors 430 and 432, and to display instructions received from an operator. Command module 450 is operable to send commands to cooling gas input valve 424 to heating gas input valve 426, and to mixed gas input valve 442, and is optionally further operable to send commands to other valves and controls of system 92.

Command module 450 is further preferably operable to algorithmically select or generate commands to be sent to gas input valve 424, to heating gas input valve 426, and to mixed gas input valve 442, such commands being based on algorithmic evaluations of data received from sensors 430 and 432, and further based on instructions received from an operator. Algorithms thus used may be stored in memory 452.

Command module 450 is further operable to record in memory 452, for later display and analysis, data received from sensors 430 and 432 and instructions received from an operator.

In system 92, command module 450 is further operable to send commands to fluid pump 470, and thus to control inflow and outflow of fluid to and from fluid transfer lumen 312. Thus, by controlling flow of fluid into and out of fluid transfer lumen 312, command module 450 is operable to control pressure within exterior volume 314 of cryoprobe 300, and thereby to control inflation and deflation of shape-adaptable treatment head 330 of cryoprobe 300. Control module 450 preferably controls inflation and deflation of head 330 under algorithmic control, according to pre-set programmed instructions, or according to instructions received from an operator in real time.

In a preferred use, command module 450 is operable to respond to instructions from an operator by adjusting flow from a plurality of gas sources, to produce a mixture which, when expanded in a Joule-Thomson orifice, will produce a selected degree of cooling. As noted hereinabove, selected steps in a therapeutic process of treatment of atrial arrhythmia may require selected degrees of cooling during different phases of a treatment process. Command module 450 is preferably operable to deliver to gas input lumen 130 a selected mixture of gas such as will produce a selected degree of cooling in treatment head 330. In a preferred embodiment, command module 450 is operable to deliver this selected mixture of gas according to a pre-selected mixture of cooling gas and of heating gas. In a further preferred embodiment, command module 450 is operable to deliver this selected mixture of gas according to algorithmically selected commands to gas input valves 424, 426, and 442, in response to temperature and pressure data receive from sensors 430 and 432.

An alternate preferred embodiment of gas supply module 460 (not shown) presents a plurality of mixed gas sources 440, (e.g., 440A, 440B, etc.), each operable to supply a mixture of heating gas and cooling gas in a selected proportion. Preferably, each of mixed gas sources 440 presents a mixture operable to supply a desired degree of cooling for a particular phase of treatment of arrhythmia, as described hereinabove.

Attention is now drawn to FIG. 17, which is a simplified schematic of a system for cryosurgery comprising a cryoprobe having an elongated head, according to a embodiment of the present invention.

System 93, illustrated by FIG. 17, is particularly recommended for treating atrial arrhythmia, and in particular for forming a conduction block in a wall of an atrium of a heart.

System 93 comprises a cryoprobe 400 having an elongated treatment head, as described hereinabove with particular reference to FIG. 12. System 93 further comprises a gas supply module 460 and a command module 450.

Gas supply module 460 is operable to supply compressed gas to gas input lumen 130 of cryoprobe 400.

Gas supply module 460 comprises a cooling gas source 420, which is a source of compressed cooling gas, and a heating gas source 422, which is a source of compressed heating gas. Flow of gas from cooling gas source 420 is controlled by cooling gas input valve 424, which is preferably a remotely controllable valve. Flow of gas from heating gas source 422 is controlled by heating gas input valve 426, which is preferably a remotely controllable valve. Gas supply module 450 further comprises one-way valves 428.

Gas supply module 460 optionally further comprises a mixed gas source 440, which is a source of a mixture of cooling gas and heating gas in selected proportion. Flow of gas from mixed gas source 440 is controlled by fixed gas input valve 442, which is preferably a remotely controllable valve.

Gas supply module 460 further optionally comprises a heat-exchanging configuration 124, operable to pre-cool cooling gas flowing towards gas input lumen 130 by transferring heat from cooling gas flowing towards gas input lumen 130 to cold cooling gas exhausting from gas exhaust lumen 132.

Heat exchanging configuration 124 is further operable to pre-heat heating gas, by transferring heat from hot heating gas exhausting from gas exhaust lumen 132, which has been heated by expansion, to compressed heating gas flowing towards gas input lumen 130.

Gas supply module 460 may further comprise other optional means to cool cooling gas flowing towards gas input lumen 130, and to heat heating gas flowing towards gas input lumen 130.

Command module 450 is operable to receive real-time data from one or more optional thermal sensors 430 and one or more optional pressure sensors 432. Thermal sensor 430 may be a thermocouple, or other form of heat sensor.

Thermal sensors 430 and pressure sensors 432 may be situated within treatment head 410 of cryoprobe 400, as illustrated in FIG. 17, or alternatively maybe be situated in shaft 160 of cryoprobe 400, or further alternatively may be situated at various points within gas supply module 450.

Thermal sensors 430 are operable to communicate temperature data to command module 450 in real time. Pressure sensors 432 are also operable to communicate temperature data to command module 450 in real time.

Command module 450 is operable to receive data from thermal sensors 430 and from pressure sensors 432. Command module 450 is further operable to receive instructions from an operator. Command module 450 preferably comprises a memory 452 and a display 454. Command module 450 is preferably operable to display data received from sensors 430 and 432, and to display instructions received from an operator. Command module 450 is operable to send commands to cooling gas input valve 424 to heating gas input valve 426, and to mixed gas input valve 442, and is optionally further operable to send commands to other valves and controls of system 93.

Command module 450 is further preferably operable to algorithmically select or generate commands to be sent to gas input valve 424, to heating gas input valve 426, and to mixed gas input valve 442, such commands being based on algorithmic evaluations of data received from sensors 430 and 432, and further based on instructions received from an operator. Algorithms thus used may be stored in memory 452.

Command module 450 is further operable to record in memory 452, for later display and analysis, data received from sensors 430 and 432 and instructions received from an operator.

In a preferred use, command module 450 is operable to respond to instructions from an operator by adjusting flow from a plurality of gas sources, to produce a mixture which, when expanded in a Joule-Thomson orifice, will produce a selected degree of cooling. As was noted hereinabove, selected steps in a therapeutic process of treatment of atrial arrhythmia may require selected degrees of cooling during different phases of a treatment process. Command module 450 is preferably operable to deliver to gas input lumen 130 a selected mixture of gas such as will produce a selected degree of cooling in treatment head 410. In a preferred embodiment, command module 450 is operable to deliver this selected mixture of gas according to a pre-selected mixture of cooling gas and of heating gas. In a further preferred embodiment, command module 450 is operable to deliver this selected mixture of gas according to algorithmically selected commands to gas input valves 424, 426, and 442, in response to temperature and pressure data receive from sensors 430 and 432.

An alternate preferred embodiment of gas supply module 460 (not shown) presents a plurality of mixed gas sources 440, (e.g., 440A, 440B, etc.), each operable to supply a mixture of heating gas and cooling gas in a selected proportion. Preferably, each of mixed gas sources 440 presents a mixture operable to supply a desired degree of cooling for a particular phase of treatment of arrhythmia, as described hereinabove.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A form-fitting cryoprobe having a treatment head sized and formed to fit a shape of a specific organic cryoablation target, said treatment head comprising a Joule-Thomson cooler operable to cool said treatment head.

2. The cryoprobe of claim 1, further operable to use Joule-Thomson heating to heat said treatment head.

3. A shape-adaptable cryoprobe having a treatment head operable to conform to a shape of a cryoablation target, said treatment head comprising a Joule-Thomson cooler operable to cool said treatment head.

4. The cryoprobe of claim 3, further operable to use Joule-Thomson heating to heat said treatment head.

5. A cryoprobe for cryogenic treatment of cardiac arrhythmia, said cryoprobe comprising:
a) a form-fitting treatment head sized and shaped to fit a pulmonary vein ostium;
b) a Joule-Thomson cooler operable to cool said treatment head.

6. The cryoprobe of claim 5, further comprising a Joule-Thomson heater operable to heat said treatment head.

7. The cryoprobe of claim 5, further comprising
c) a gas input lumen operable to supply compressed cooling gas to said treatment head; and
d) a gas exhaust lumen operable to exhaust gas from said treatment head.

8. The cryoprobe of claim 7, further comprising a plurality of gas input lumens.

9. The cryoprobe of claim 8, wherein supply of gas to each of said plurality of gas input lumens is operable to be individually controlled.

10. The cryoprobe of claim 5, wherein said treatment head further comprises a Joule-Thomson orifice.

11. The cryoprobe of claim 5, further comprising a heat exchanging configuration.

12. The cryoprobe of claim 5, further comprising an active cooling module on a distal face of said treatment head.

13. The cryoprobe of claim 12, wherein said active cooling module is operable to create a temporary conduction block in a pulmonary vein ostium.

14. The cryoprobe of claim 12, wherein said active cooling module is operable to create a permanent conduction block in a pulmonary vein ostium.

15. The cryoprobe of claim 12, wherein said active cooling module is operable to create a temporary conduction block in a pulmonary vein ostium, and further operable to create a permanent conduction block in a pulmonary vein ostium.

16. The cryoprobe of claim 12, wherein said active cooling module is further operable to heat tissues of a pulmonary vein ostium.

17. The cryoprobe of claim 12, further comprising a plurality of active cooling modules on said distal face of said treatment head.

18. The cryoprobe of claim 17, wherein said plurality of active cooling modules are radially distributed.

19. The cryoprobe of claim 17, wherein said plurality of active cooling modules are circumferentially distributed.

20. The cryoprobe of claim 17, wherein each of said plurality of active cooling modules is in fluid communication with an independently controlled source of cooling gas.

21. The cryoprobe of claim 20, wherein supply of gas to each of a plurality of gas input lumens is operable to be individually controlled.

22. The cryoprobe of claim 12, wherein said active cooling module comprises a heat-conductive surface operable to conduct heat between said cooling module and tissues of a body.

23. The cryoprobe of claim 5, further comprising a flexible shaft attached to said treatment head.

24. The cryoprobe of claim 23, wherein said flexible shaft comprises flexibly attached rigid segments.

25. The cryoprobe claim 5, wherein said cryoprobe further comprises a sensor operable to transmit data to a control module external to said cryoprobe.

26. The cryoprobe of claim 25, wherein said sensor is operable to transmit data over a wire.

27. The cryoprobe of claim 25, wherein said sensor is operable to transmit data by wireless transmission.

28. The cryoprobe of claim 25, wherein said sensor is a thermal sensor.

29. The cryoprobe of claim 25, wherein said sensor is a pressure sensor.

30. The cryoprobe of claim 25, further comprising a plurality of sensors operable to transmit data to a control module external to said cryoprobe.

31. The cryoprobe of claim 30, wherein at least one of said plurality of sensors is a thermal sensor and at least one of said plurality of sensors is a pressure sensor.

32. A shape-adaptable cryoprobe, having a treatment head operable to adaptively conform to a shape of an organic target, thereby enhancing transfer of heat between said treatment head and said organic target.

33. The cryoprobe of claim 32, wherein said treatment head is operable to adaptively conform to a shape of a pulmonary vein ostium.

34. The cryoprobe of claim 32, wherein said treatment head is inflatable.

35. The cryoprobe of claim 32, wherein said treatment head is operable to be cooled by Joule-Thomson cooling.

36. The cryoprobe of claim 32, wherein said treatment head comprises a Joule-Thomson orifice.

37. The cryoprobe of claim 32, wherein said treatment head is operable to be heated by Joule-Thomson heating.

38. The cryoprobe of claim 32, wherein said treatment head comprises an expandable volume defined by a flexible inflatable external sleeve.

39. The cryoprobe of claim 38, wherein said expandable volume is operable to be cooled by expanding cooling gas flowing into said expandable volume through a Joule-Thomson orifice.

40. The cryoprobe of claim 34, wherein said treatment head comprises a Joule-Thomson cooler.

41. The cryoprobe of claim 32, further comprising:
a) a gas input lumen for supplying a pressurized cooling gas;
b) a Joule-Thomson orifice at a termination of said gas input lumen; and
c) a flexible inflatable external sleeve operable to be inflated by gas passed through said Joule-Thomson orifice.

42. The cryoprobe of claim 41, further comprising:
d) a gas exhaust lumen for exhausting gas from said treatment head; and
e) a gas exhaust valve operable to control flow of gas through said gas exhaust lumen.

43. The cryoprobe of claim 32, further comprising an inner cooling module operable to be cooled by a Joule-Thomson cooler, and an exterior expansion volume defined within a flexible inflatable exterior sleeve, said exterior expansion volume being exterior to said inner cooling module.

44. The cryoprobe of claim 43, wherein said inner cooling module comprises a Joule-Thomson orifice.

45. The cryoprobe of claim 43, further comprising a fluid transfer lumen, a gas input lumen, and a gas exhaust lumen.

46. The cryoprobe of claim 43, wherein said expansion volume is in fluid communication with said fluid transfer lumen.

47. The cryoprobe of claim 43, wherein said expansion volume is operable to expand when filled by a fluid supplied under pressure through said fluid transfer lumen.

48. The cryoprobe of claim 43, wherein said inner cooling module is operable to cool a fluid within said expansion volume.

49. A linear cryoprobe operable to apply cryogenic cooling to body tissues in an elongated pattern, comprising:
a) a treatment head comprising a Joule-Thomson orifice and a heat-conducting surface so shaped that a ratio of length of said surface to width of said surface is greater than six to one;
b) a gas input lumen; and
c) a gas exhaust lumen;

50. The cryoprobe of claim 49, wherein said treatment head further comprises an insulating shroud.

51. A system for treating cardiac arrhythmia, comprising
a) a control module operable to receive data from a sensor;
b) a cryoprobe which comprises:
i) a treatment head comprising a Joule-Thomson orifice; and
ii) a gas input lumen operable to supply a pressurized gas to said Joule-Thomson orifice; and
b) a gas supply module operable to supply compressed gas to said gas input lumen.

52. The system of claim 51, wherein said cryoprobe further comprises a cryoprobe sensor operable to transmit data to said control module.

53. The system of claim 52, wherein said sensor is operable to transmit data to said control module by wireless communication.

54. The system of claim 52, wherein said cryoprobe further comprises a plurality of cryoprobe sensors operable to transmit data to said control module.

55. The system of claim 52, wherein said cryoprobe sensor is a thermal sensor.

56. The system of claim 52, wherein said cryoprobe sensor is a pressure sensor.

57. The system of claim 54, wherein at least one of said plurality of sensors is a thermal sensor and at least one of said plurality of sensors is a pressure sensor.

58. The system of claim 51, wherein said gas supply module comprises a plurality of sources of compressed gas.

59. The system of claim 58, wherein said plurality of sources comprises a source of compressed cooling gas.

60. The system of claim 58, wherein said plurality of sources comprises a source of compressed heating gas.

61. The system of claim 58, wherein said plurality of sources comprises a source of mixed cooling gas and heating gas.

62. The system of claim 61, wherein said plurality of sources comprises a plurality of sources of mixed cooling gas and heating gas.

63. The system of claim 51, further comprising a cooling gas input valve controlling flow of cooling gas from said gas supply module into said gas input lumen.

64. The system of claim 63, wherein said cooling gas input valve is controllable by commands transmitted by said control module.

65. The system of claim 63, further comprising a heating gas input valve controlling flow of heating gas from said gas supply module into said gas input lumen.

66. The system of claim 65, wherein said heating gas input valve is controllable by commands transmitted by said control module.

67. The system of claim 51, wherein said gas supply module comprises a heat exchanging configuration.

68. The system of claim 51, wherein said cryoprobe comprises a heat-exchanging configuration.

69. The system of claim 51, wherein said cryoprobe comprises a treatment head sized and shaped to fit a pulmonary vein ostium.

70. The system of claim 51, wherein said cryoprobe comprises a treatment head operable to adaptively conform to a shape of an organic target, thereby enhancing transfer of heat between said treatment head and said organic target.

71. The system of claim 70, wherein said cryoprobe is operable to adaptively conform to a shape of a pulmonary vein ostium.

72. The system of claim 51, wherein said treatment head is inflatable.

73. The system of claim 72, wherein said inflatable treatment head comprises a Joule-Thomson orifice.

74. The system of claim 51, wherein said cryoprobe is operable to apply cryogenic cooling to body tissues in an elongated pattern.

75. The system of claim 74, wherein said cryoprobe comprises:
a) a treatment head which comprises a Joule-Thomson orifice and a heat-conducting surface so shaped that a ratio of length of said surface to width of said surface is greater than six to one;
b) a gas input lumen; and
c) a gas exhaust lumen.

76. A method for treating cardiac arrhythmia, comprising:
a) introducing a cryoprobe into an atrium of a heart;
b) positioning said cryoprobe at an ostium of a pulmonary vein, in such a position that an active cooling module of said cryoprobe is in contact with tissues of said ostium;
c) cooling said active cooling module to a first temperature, said first temperature being such as to cause said cryoprobe to adhere to tissues of said ostium, thereby causing said cryoprobe to adhere to said tissues of said ostium;
d) testing said positioning of said cryoprobe by cooling said active cooling module to a second temperature, said second temperature being such as to create a temporary conduction block in said ostium if said cryoprobe is correctly positioned, thereby creating a temporary conduction block in said ostium if said cryoprobe is correctly positioned;
e) evaluating said positioning of said cryoprobe by determining whether said temporary conduction block was created by step (d);
f) if said temporary conductive block was created by step (d), cooling said active cooling module to a third temperature, said third temperature being such as to create a permanent conductive block in said ostium, thereby creating a permanent conductive block in said ostium, thereby treating said cardiac arrhythmia.

77. The method of claim 76, further comprising
g) heating said cryoprobe to free said cryoprobe from said adhesion if a conductive block is not created by step (d); and
h) repositioning said cryoprobe at said ostium.

78. The method of claim 76, further comprising:
i) heating said cryoprobe after cooling said active cooling module to said third temperature, thereby releasing said cryoprobe from said adhesion after having created said conductive block.

79. The method of claim 76, wherein said cryoprobe is sized and formed to conform to a shape of a pulmonary vein ostium.

80. The method of claim 76, wherein said cryoprobe comprises an inflatable portion, and is operable to adaptively conform to a shape of a pulmonary vein ostium.

81. The method of claim 80, further comprising
j) endoscopically introducing said cryoprobe into an atrium;
k) introducing a distal portion of said cryoprobe into an opening of a pulmonary vein; and
l) inflating said inflatable portion;
thereby adaptively conforming said cryoprobe a shape of said pulmonary vein ostium.

82. A method for treating cardiac arrhythmia, comprising:
a) positioning at an exterior wall of a atrium a cryoprobe having a treatment head which comprises an elongated cooling surface;
b) cooling said cooling surface to a first temperature, said first temperature being such as to cause said cryoprobe to adhere to tissues of said atrium wall, thereby causing said cryoprobe to adhere to tissues of said atrium wall;
c) testing said positioning of said cryoprobe by cooling said cooling surface to a second temperature, said second temperature being such as to create a temporary conduction block in said atrium wall if said cryoprobe is correctly positioned, thereby creating a temporary conduction block in said atrium wall if said cryoprobe is correctly positioned;
d) evaluating said positioning of said cryoprobe by determining whether said temporary conduction block was created by step (d);
e) if said temporary conduction block was created by step (d), cooling said active cooling module to a third temperature, said third temperature being such as to create a permanent a permanent conduction block in said atrium wall, thereby creating a permanent conduction block in said atrium wall, thereby treating said cardiac arrhythmia.
